(19) United States
(12) Patent Application Publication
(10) Pub. No.: US 2019/0328658 A1
(43) Pub. Date: Oct. 31, 2019

(54) ESTROGEN PRODRUGS AND METHODS OF ADMINISTERING ESTROGEN PRODRUGS

(71) Applicant: Evestra, Inc., Schertz, TX (US)

(72) Inventors: Klaus Nickisch, Berlin (DE); Karin Eggenreich, Schertz, TX (US); Simone Eder, Graz (AT); Andreas Witschnigg, Villach (AT)

(21) Appl. No.: 16/395,349

(22) Filed: Apr. 26, 2019

Related U.S. Application Data
(60) Provisional application No. 62/663,584, filed on Apr. 27, 2018.

(51) Int. Cl. A61K 9/00; A61K 9/02; A61K 31/565; A61K 31/567; A61K 31/57; A61K 47/32; A61K 47/34

(52) U.S. Cl. CPC A61K 9/0036; A61K 9/025; A61K 31/565; A61K 47/34; A61K 31/57; A61K 47/32; A61K 31/567

(57) ABSTRACT

Disclosed herein is an intravaginal drug delivery device that includes one or more compartments, each of the one or more compartments comprising an estrogen prodrug and/or a progestin dispersed in a thermoplastic polymeric matrix.

### PRIORITY CLAIM

This application claims priority to U.S. Provisional Application Ser. No. 62/663,584 entitled “TARGETED DELIVERY OF PROGESTINS AND ESTROGENS VIA VAGINAL RING DEVICES FOR FERTILITY CONTROL AND HRT PRODUCTS” filed Apr. 27, 2018, which is incorporated herein by reference in its entirety.

### BACKGROUND OF THE INVENTION

### 1. Field of the Invention

The invention generally relates to the use of estriol prodrugs as active ingredients for the production of vaginal rings, for the treatment of climacteric complaints, for the prevention of osteoporosis as single agent, and in combination with a progestin as contraceptive

### 2. Description of the Relevant Art

The reduced production of estrogens after menopause can lead to phenomena that require therapy. Hormone replacement with natural estrogens like estradiol or estrone quickly leads to an improvement in climacteric symptoms like hot flushes and night sweats. In addition, such treatment can prevent advancing osteoporosis.

Against such benefits stand some risks such as the growth of hormone dependent tumors and deep vein thrombosis.

The situation is different for estriol. In a large observational study, oral estriol was not associated with a risk of breast cancer. In addition, estriol has not been used in combination with progestins for contraception.

Estriol (“E3”) seems to be ideally suited for these indications based on its different pharmacological profile compared to estradiol and, especially, ethinyl estradiol. For example, estriol does stimulate uterine weights when administered once to ovariectomized rats. In combination with other strong estrogens like estradiol, it even acts as an anti-estrogen, blocking the stimulatory effect of estradiol. Estriol has been widely used after vaginal administration in application form of tablets or ovula or creams and ointments for the local treatment of vaginal atrophy.

Vaginal or oral application of estriol leads to fast absorption, but the high clearance leads to a fast elimination so that after 4-6 hours after application no estriol plasma level above the limit could be determined.

A drug delivery system, like a vaginal ring, that would lead to constant, therapeutically relevant plasma levels would be therefore highly desirable for the treatment of climacteric symptoms and osteoporosis in postmenopausal women and in combination with progestins as contraceptive agents.

Estriol has been used for the local therapy of certain menopausal symptoms. In U.S. Patent Application Publication No. 2011/0086825 a topical formulation is described which includes progesterone, testosterone and estriol.

PCT Publication No. WO 2009/000954 describes the use of low dose estriol for the treatment/prevention of vaginal atrophy. U.S. Patent Application Publication No. 2011/0312929 describes an estriol formulation with the capacity to self-limit the absorption of estriol for the treatment of urogenital atrophy, and in PCT Publication No. WO 2010/069621 the treatment of vaginal atrophy for women with a cardiovascular risk is described.

A film based estriol oral formulation for the buccal application of estriol is described in PCT Publication No. WO 2005/110358 by Elger et al. for the treatment of climacteric symptoms. The same group describes in U.S. Pat. No. 5,614,213 a transdermal product that releases estriol over 24 hours.

Estriol derivatives have been described in U.S. Pat. No. 4,780,460, in which glycol esters of estriol have been described in order to form an aqueous crystalline suspension.

In U.S. Pat. No. 4,681,875 3,17-estriol esters were disclosed for the prolonged subcutaneous application of estriol. Estriol esters were also disclosed in U.S. Pat. No. 6,894,038 for the treatment of autoimmune diseases such as multiple sclerosis.

It can be concluded that no approach has been described to generate long-lasting therapeutic plasma levels of estriol that would be needed in order to treat climacteric symptoms and to provide activity in the prevention of osteoporosis.

### SUMMARY OF THE INVENTION

In an embodiment, an intravaginal drug delivery device includes one or more compartments, each of the one or more compartments comprising an estrogen prodrug and/or a progestin dispersed in a thermoplastic polymeric matrix. In some embodiments, one or more of the compartments are uncoated compartments. In some embodiments, one or more of the compartments are coated compartments comprising an estrogen prodrug and/or progestin dispersed in a coated thermoplastic polymeric matrix. In some embodiments, the device comprises two or more compartments having different sizes.

In an embodiment, the estrogen prodrug is a mono or di ester of estriol. Alternatively, the estrogen prodrug includes a prodrug of estriol having structure:

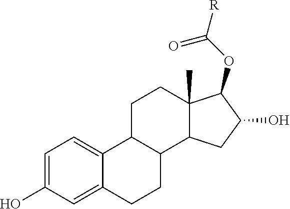

where R is a saturated hydrocarbon. In some embodiments, R is methyl. In some embodiments, R is cyclopropyl.

In an embodiment, the estrogen prodrug is a mono or di ester of estriol. Alternatively, the estrogen prodrug includes a prodrug of estriol having structure:

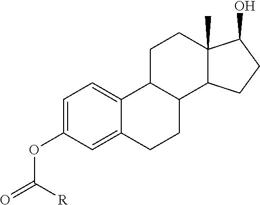

where R is a saturated hydrocarbon. In some embodiments, R is methyl. In some embodiments, R is cyclopropyl.

In an embodiment, the estrogen prodrug is a mono or di ester of estriol. Alternatively, the estrogen prodrug includes a prodrug of estriol having structure:

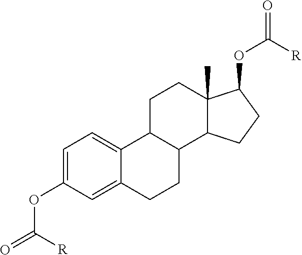

where R is a saturated hydrocarbon. In some embodiments, R is cyclopropyl.

In an embodiment, the device includes at least one compartment containing a progestin, and wherein the progestin is released, during vaginal use, in an amount sufficient to inhibit ovulation in fertile women. In some embodiments, the progestin is trimegestone.

In an embodiment, the device comprises at least one compartment that includes an estrogen prodrug and at least one compartment that includes a progestin, and wherein the estrogen prodrug and progestin are released, during vaginal use, in amounts sufficient to effect cycle control in fertile women. In an embodiment, the intravaginal drug delivery device provides the estrogen prodrug and/or the progestin according to a non-zero order release profile.

In an embodiment, the thermoplastic polymeric matrix comprises an ethylene vinyl acetate copolymer. In an embodiment, the thermoplastic polymeric matrix comprises a thermoplastic polyurethane. In an embodiment, the compartment is a coated compartment that includes a thermoplastic polymeric matrix comprising an ethylene vinyl acetate (EVA) copolymer with a VA (vinyl acetate) content between 18% and 40% and wherein the coating comprises an EVA copolymer with a VA content between 6% and 18%. In another embodiment, the thermoplastic polymeric matrix includes an ethylene vinyl acetate (EVA) copolymer with a VA (vinyl acetate) content between 18% and 40% in the core and a low-density polyethylene (LDPE).

In an embodiment, the device has a substantially annular form. In an embodiment, the device has a cross-sectional diameter in the range of 3.8-8.0 mm. In an embodiment, the device has an outer diameter in the range of 52-58 mm.

In an embodiment, the device delivers an effective amount of the progestin and the estrogen prodrug for at least 21 days. In an embodiment, the amount of progestin and/or estrogen prodrug released by the device on the last day of treatment is at least 50% higher than on any day after the first day of use.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts the in vitro release rates of various estriol prodrugs synthesized according to the present description; and

FIG. 2 depicts a graph of plasma levels of various estriol prodrugs in sheep.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

Examples provided herein are included to demonstrate preferred embodiments of the invention. It should be appreciated by those of skill in the art that the techniques disclosed in any of the examples disclosed herein represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Estriol Prodrugs

In an embodiment, an estriol prodrug has the structure:

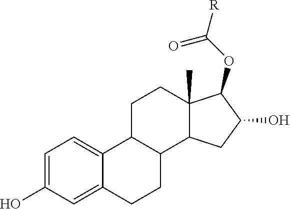

where R is a saturated hydrocarbon. In specific embodiments, R may be either methyl or cyclopropyl.

The term “hydrocarbon” as used herein generally refers to a chemical substituent containing only carbon and hydrogen. In some embodiments, hydrocarbons include molecules having the formula CₙH₂ₙ, where n is an integer greater than zero. In some embodiments n is 1 to 12. The term “hydrocarbon” includes a branched or unbranched monovalent hydrocarbon radicals. Examples of hydrocarbon radicals include, but are not limited to: methyl, ethyl, propyl, isopropyl, butyl, iso-butyl, sec-butyl, pentyl, 3-pentyl, hexyl, heptyl, octyl, nonyl, decyl, undecyl, dodecyl. When the alkyl group has from 1-6 carbon atoms, it is referred to as a “lower alkyl.” Suitable lower alkyl radicals include, but are not limited to, methyl, ethyl, n-propyl, i-propyl, 2-propenyl (or allyl), n-butyl, t-butyl, and i-butyl (or 2-methylpropyl). The term “hydrocarbon” also encompasses cyclic hydrocarbons such as, for example, cyclopropyl, cyclobutyl, cyclopentyl and cyclohexyl.

In another embodiment, an estriol prodrug has the structure:

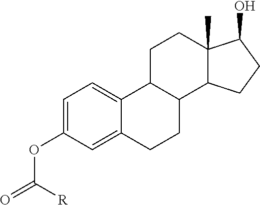

where R is a saturated hydrocarbon. In specific embodiments, R may be either methyl or cyclopropyl.

In another embodiment, an estriol prodrug has the structure:

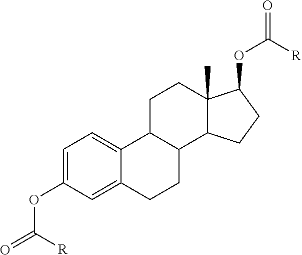

where R is a saturated hydrocarbon. In specific embodiments, R may be cyclopropyl.

### EXAMPLES

### Example 1: Synthesis of (16R,17R)-3,16-dihydroxy-7,8,9,11,12,13,14,15,16,17-decahydro-6H-cyclopenta α phenanthren-17-yl cyclopropanecarboxylate (EC537)

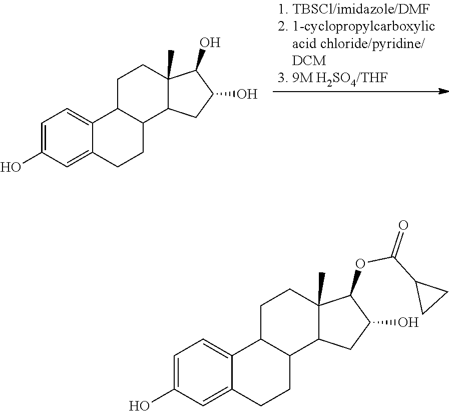 [SMILES: C[C@]12CCC3c4ccc(O)cc4CCC3C1C[C@@H](O)[C@@H]2O ; C[C@]12CCC3c4ccc(O)cc4CCC3C1C[C@@H](O)[C@@H]2OC(=O)C1CC1]

An oven-dried 3-necked flask was charged with steroid (75 g, 0.26 mol) and imidazole (70.7 g, 1.04 mol). DMF was added, and a solution was allowed to form before the addition of TBSCl (97.6 g, 0.65 mol). The mixture was allowed to stir for 40 minutes, after which time the reaction was judged complete by TLC. The mixture was then diluted with 1.2 L of ice-water, and then extracted with MTBE (700 ml). The organic layer was washed with water (2×500 ml). The combined aqueous layers were extracted with MTBE (2×350 ml). The combined organic layers were washed with brine and dried over sodium sulfate. 158.42 g of crude was obtained after removal of the ether, and this was subjected to flash chromatography using 2.75 kg of silica gel, and a 3.5 to 7% gradient of ethyl acetate in hexanes. 118.84 g of intermediate 1 was obtained (88% yield).

Intermediate 1 (277.4 g, 0.54 mol) was dissolved in DCM (1.4 L) and pyridine (260 ml, 3.23 mol). 1-cyclopropylcarboxylic acid chloride (58.5 ml, 0.64 mol) was added dropwise and the mixture was allowed to stir overnight following which time TLC indicated complete consumption of starting material. The volatiles were then removed (heptane was added to remove pyridine), leaving behind 315.1 g of crude solid which was then stirred in 1.5 L of 10% aqueous methanol for 1 hour and then filtered, leaving 304.7 g of intermediate 2 in 97% yield.

Intermediate 2 (216.1 g, 0.37 mol) was dissolved in THF (1.1 L), and 400 ml of 9 M sulfuric acid was added dropwise. When the reaction was judged complete, the mixture was diluted with 5.5 L of water, and then extracted with ethyl acetate (600 ml, and then 2×300 ml). The combined organics were washed with saturated sodium bicarbonate solution, brine, and dried over sodium sulfate. The crude material was then subjected to flash chromatography using first DCM, and then after three column volumes 10% acetone in DCM. 117 g of product was obtained (89% yield). This was then crystallized from acetone-hexanes to produce the title compound—¹H NMR (δ, CDCl₃, 300 MHz): 7.15 (d, J=8.4 Hz, 1H), 6.65 (dd, J=8.6 Hz, 2.4 Hz, 1H), 6.58 (s, 1H), 5.14 (s, 1H), 4.27 (d, J=4.5 Hz, 1H), 4.19-1.16 (m, 1H), 3.98 (s, 1H), 0.98 (s, 3H). IR (cm−1): 3378, 3324, 3148, 2921, 2845, 1733, 1171. Melting Point: 160.0-166.5° C.

### Example 2: Synthesis of (8xi,9xi,14xi,16α,17β)-3,17-dihydroxyestra-1(10),2,4-trien-16-yl cyclopropanecarboxylate

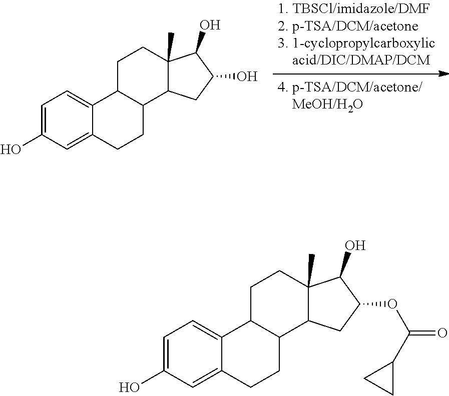 [SMILES: C[C@]12CCC3c4ccc(O)cc4CCC3C1C[C@@H](O)[C@@H]2O ; C[C@]12CCC3c4ccc(O)cc4CCC3C1C[C@@H](OC(=O)C1CC1)[C@@H]2O]

An oven-dried 3-necked flask was charged with estriol (40 g, 0.14 mol) and imidazole (40 g, 0.58 mol). DMF was added (1 L), and a solution was allowed to form before the addition of TBSCl (80 g, 0.53 mol). The mixture was allowed to stir for 40 minutes, after which time the reaction was judged complete by TLC. The mixture was then diluted with 1.6 L of ice-water, and then extracted with ether (3×300 ml). The organic layers were washed with water (3×200 ml), brine and dried over sodium sulfate. 105.5 g of crude was obtained after removal of the ether, and this was subjected to flash chromatography using 1.5 kg of silica gel, and 2% ethyl acetate in hexanes, leading to 50.23 g of 3,17 (tri. butyl silyloxy) 16 alpha estradiol as intermediate 1 (58% yield).

DIC (6.9 mL, 0.044 mol) was added to cyclopropanecarboxylic acid (3.9 mL, 0.49 mol) in DCM (20 mL) at room temp. After 30 min of stirring, estriol intermediate 1 (10.5 g, 0.02 mol) in DCM (30 mL) was added and followed by the addition of DMAP (124 mg, 1.0 mmol). The resulting white slurry mixture was stirred at rt for 24 hours. TLC showed complete consumption of the starting material. The residue was purified by silica gel chromatography using 5% ethyl acetate in hexanes as eluent to afford the ester intermediate 2 (11.2 g, 94% yield).

Intermediate 2 (11.2 g, 18.8 mmol) was dissolved in DCM (65 mL), acetone (65 mL), methanol (9 mL) and water (3 mL, 12 equivalents) at room temp, followed by addition of pTsOH (5.36 g, 28.2 mmol). After 16 hours of stirring, one more equivalent (3.64 g, 18.8 mmol) of pTsOH was added and the resulting solution was stirred for 7 hours. TLC showed complete consumption of the starting material. The product was purified by silica gel chromatography using 20-30% ethyl acetate in hexanes as eluent to afford 5.3 g of the ester of the title compound (79% yield).

¹H NMR (δ, CDCl₃ 300 MHz): 7.11 (d, J=8.4 Hz, 1H), 6.61 (dd, J=8.4, 3.0 Hz, 1H), 6.54 (d, J=3.0 Hz, 1H), 4.81 (dddd, J=13.8, 8.7, 4.8, 1.8 Hz, 1H), 3.60 (d, J=4.8 Hz, 1H), 2.82 (m, 2H), 2.52 (m, 2H), 0.84 (s, 3H). IR (cm⁻¹): 3508, 3290, 1692, 1603, 1502, 1451, 1402, 1198, 1039, 913, 824, 649. Melting Point: 186.8-187.9° C.

### Example 3: (8xi,9xi,14xi,16α,17β)-17-hydroxyestra-1(10),2,4-triene-3,16-diyl diacetate

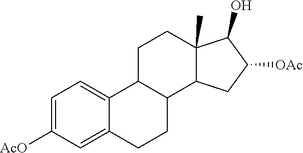 [SMILES: CC(=O)Oc1ccc2c(c1)CCC1C2CC[C@@]2(C)C1C[C@@H](OC(C)=O)[C@@H]2O]

10 g (34.7 mmol) of estriol was suspended in DCM (250 ml) and pyridine (45 ml, 0.56 mol), and chilled in an ice bath. Acetyl chloride (5.18 ml, 72.8 mmol) was then added dropwise over 45 min. The mixture was washed with water, then brine. Heptane was added to rid of pyridine under vacuum. The crude mixture was then subjected to silica gel chromatography using 400 g of silica gel in 5% acetone/DCM to obtain 4.75 g of product. This was crystallized from acetone-hexanes to obtain the final product.

¹H NMR (δ, CDCl₃, 300 MHz) 7.30-7.26 (m, 1H), 6.85 (dd, J=8.4, 2.4 Hz, 1H), 6.79 (d, J=2.7 Hz, 1H), 4.83 (dddd, J=13.8, 10.8, 5.1, 1.8 Hz, 1H), 3.64-3.61 (m, 1H), 3.37 (d, J=2.1 Hz, 1H), 2.89-2.84 (m, 2H), 2.29 (s, 3H), 2.11 (s, 3H), 0.86 (s, 3H). IR (cm⁻¹): 3496, 1751, 1712, 1704, 1375, 1270, 1198, 1172, 1033, 868. Melting Point: 139.6-140.1° C.

### Example 4: (8xi,9xi,14xi,16α,17β)-16,17-dihydroxyestra-1(10),2,4-trien-3-yl acetate (EC5104)

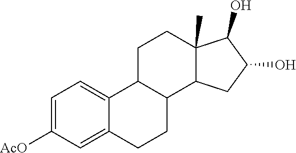 [SMILES: CC(=O)Oc1ccc2c(c1)CCC1C2CC[C@@]2(C)C1C[C@@H](O)[C@@H]2O]

100 g of estriol (0.34 mol) was suspended in 2 propanol (1.5 L) and then 690 ml of 2 M NaOH was added, and the thick slurry was allowed to stir for ten minutes before addition of acetic anhydride (130 ml, 1.38 mol). The now homogenous mixture was then diluted with 4 L of 4% potassium bicarbonate and the resulting solids collected by vacuum filtration and the solids were allowed to dry on the filter overnight. The next day the solids were taken up into boiling acetone (2 L), the mixture allowed to cool, and then filtered. The solvent from the filtrate was then distilled off to dry the material. The resulting solids were then crystallized from acetone to produce the title compound (62 g, 57% yield). ¹H NMR (δ, CDCl₃, 300 MHz) 7.29-7.26 (m, 1H), 6.84 (dd, J=8.4, 2.4 Hz, 1H), 6.79 (d, J=2.4 Hz, 1H), 4.18 (m, 1H), 3.60 (d, J=5.4 Hz, 1 Hz), 2.86 (m, 2H), 2.29 (s, 3H), 0.81 (s, 3H). IR (cm⁻¹): 3460, 3352, 1726, 1496, 1422, 1375, 1233, 1051, 1010, 950, 875, 821. Melting Point: 181.6-183.5° C.

### Example 5: (8xi,9xi,14xi,16α,17β)-16,17-dihydroxyestra-1(10),2,4-trien-3-yl cyclopropanecarboxylate (EC5105)

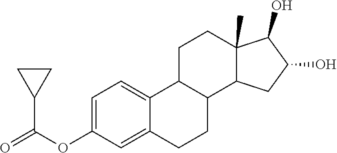 [SMILES: C[C@]12CCC3c4ccc(OC(=O)C5CC5)cc4CCC3C1C[C@@H](O)[C@@H]2O]

62.3 g of estriol (0.22 mol) was suspended in 2 propanol (1.5 L) and then 325 ml of 2 M NaOH (0.65 mol) was added, and the thick slurry was allowed to stir for ten minutes before addition of 1-cyclopropylcarboxlyic acid anhydride (100 g, 0.65 mol). The now homogenous mixture was then diluted with 4 L of 4% potassium bicarbonate and the resulting solids collected by vacuum filtration and the solids were allowed to dry on the filter overnight. The resulting solids were then crystallized from acetone-hexanes to produce the title compound. ¹H NMR (δ, CDCl₃, 300 MHz) 7.29-7.25 (m, 1H), 6.84 (dd, J=8.4, 2.4 Hz, 1H), 6.79 (d, J=2.4 Hz, 1H), 4.20-4.15 (m, 1H), 3.60 (t, J=4.8 Hz, 1 Hz), 2.88-2.82 (m, 2H), 2.35-2.23 (m, 2H), 0.80 (s, 3H). IR (cm⁻¹): 3541, 3453, 3244, 1726, 1381, 1136, 1085, 1060, 885. Melting Point: 134-137° C.

### Example 6: 3,16α,17β-Trihydroxyestra-1,3,5(10)-triene 3,17-biscyclopropane carboxylate

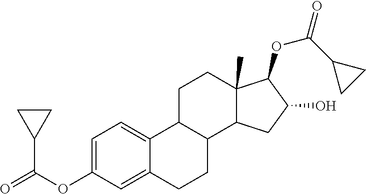 [SMILES: C[C@]12CCC3c4ccc(OC(=O)C5CC5)cc4CCC3C1C[C@@H](O)[C@@H]2OC(=O)C1CC1]

7.13 g of (16R,17R)-3,16-dihydroxy-7,8,9,11,12,13,14,15,16,17-decahydro-6H-cyclopenta[a]phenanthren-17-ylcyclopropanecarboxylate (0.02 mol) was suspended in 2 propanol (150 ml) and then 27 ml (0.054 mol) of 2 M NaOH was added, and the thick slurry was allowed to stir for ten minutes before addition of 1-cyclopropylcarboxlyic acid anhydride (8.32 g, 0.054 mol). The mixture was then extracted with DCM, which was rotovapped onto silica gel and subjected to flash chromatography using a 0 to 4% gradient of acetone in DCM and then crystallized from acetone-hexanes to give 5.0 g of the title compound in 59% yield. 1H NMR (δ, CDCl₃, 300 MHz): 7.261 (d, J=8.4 Hz, 1H), 6.84 (dd, J=8.4, 2.4 Hz, 1H), 6.792 (d, J=2.4 Hz, 1H), 4.251 (d, J=4.5 Hz, 1H), 4.140 (m, 1H), 2.870 (m, 2H), 0.883 (s, 3H). IR (cm⁻¹): 3496, 1745, 1702, 1383, 1141. Melting Point: 129.8-130.4° C.

### Example 7: General Procedure: Extrusion and Ring Closure

The estriol prodrugs were processed via hot-melt extrusion and subsequent ring closure to intravaginal rings: The EVA28 powder was dry blended with the estriol-pro drugs at a pre-defined impeller speed and time in a high shear blender to yield a homogeneous, drug loaded EVA powder blend. The hot melt extrusion line for processing the estriol containing EVA28 powder consisted of an 18 mm twin screw extruder, equipped with a loss in weight feeder for dosing the drug containing premix into the extruder. Extrusion was performed at low throughputs of approx. 2 kg/h, the temperature profile of the extruder barrels was adjusted to yield a melt temperature of approx. 125° C. After leaving the die, the strands were directly conveyed through a water bath to obtain a fast cooling process and minimize potential strand deformation. As haul-off unit, a strand pelletizer without knives was used to pull and convey the strand accurately through all downstream sections, ensuring homogeneous strand diameters and a spherical shape. The haul-off speed was adjusted to achieve a constant cross-sectional diameter of 4.0 mm. The cross-sectional diameter of the co-extrudate was measured in-line with a laser system (3 laser heads). In a final unit operation, the estriol-pro drug containing extrudates were cut into strands of appropriate length and hot air welded with a drug-free EVA28 strand of 4.0 mm cross-sectional diameter to a segmented ring, again using EVA28 as the welding material to yield IVRs with an outer diameter of 54 mm. Alternatively, the ring closure was accomplished via an injection molding process equipment, equipped with a 4.0 mm mold (one or multiple cavities), using EVA28 placebo material as injection material.

### Example 8

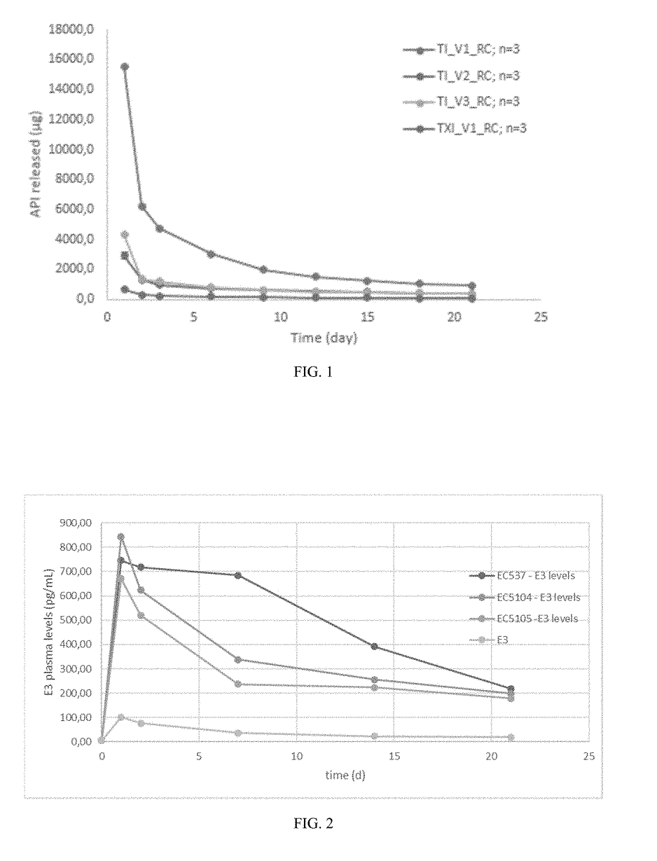

To determine the in vitro release rates, a rotational incubator, operated at 37±0.5° C., is used. The dissolution medium type, the dissolution medium volume and the rotational speed of the used incubator are selected to provide sink conditions. Samples of approx. 1 mL are withdrawn every 24 f 0.5 hours (and multiples thereof), the medium is replaced by fresh, preheated media and the samples are analyzed for their drug content via high performance liquid chromatography (HPLC) and UV/Vis detection using PDA. The results are presented in Table 1, below and in FIG. 1.

TABLE 1

| | | | | | | Average |
|---|---|---|---|---|---|---|
| | | | | | | API |
| | | API | | Burst | Burst | release |
| Abbreviation | | loading | Core | Release | Release | d3-d21 |
| (FIG. 1) | API | (%) | Polymer | d1 (μg) | d2 (μg) | (μg/day) |
| TI_V1_RC | EC537 | 5.0 | EVA28/2.5 | 15521.1 | 6229.4 | 1785.5 |
| TI_V2_RC | EC5104 | 5.0 | EVA28/2.5 | 2891.6 | 1322.0 | 548.1 |
| TI_V3_RC | EC5105 | 5.0 | EVA28/2.5 | 4301.1 | 1369.8 | 567.3 |
| TXI_V1_RC | E3 | 5.0 | EVA26/4.0 | 665.1 | 294.5 | 119.4 |

### Example 9: Plasma Levels of Estriol Prodrugs in Sheep

20 Ewes between 2 and 4 years old were checked for pregnancy and randomly divided in four groups with 5 animals each. Animals were maintained indoors under natural lightning conditions and fed a constant diet of hay, straw and oat, water and mineral licks were available ad libitum. Animals were treated with 2 injections of 0.125 mg Cloprestenol and shortly after the second injection the vaginal rings were inserted.

Blood draw occurred at days 0,1,2,7, 14 and 21 by jugular vein venipuncture directly into BD Vacutainer tubes (10 ml, LH 170 I:U). Samples were centrifuged (3000 rpm, 4 degrees for 15 min) and then blood plasma was withdrawn into Eppendorf tubes and stored at −40 degree C. Analysis was performed at ACC GmbH, Leidersbach, Germany. The results are presented in FIG. 2.

### Example 10: Determination of Log p and Permeability Values of Estriol Prodrugs

Permeability of different pro drugs has been tested at Absorption Systems, Exton Pa.

Caco-2 cells (clone C2BBel) were obtained from American Culture Collection. Cell monolayers were grown to confluence on collagen-coated, microporous membranes in 12-well assay plates. The permeability assay buffer was Hank's balanced salt solution. The buffer in the receiving chamber also contained 1% bovine serum albumin. The dosing solution concentration was 5myMof test article in the assay buffer. Cell monolayers were dosed on the apical side (A-to-B) and incubated at 37° C. with 5% CO₂ in a humidified incubator. Samples were taken from the donor and receiver chamber at 120 minutes. All samples were assayed by LC-MS/MS using electrospray ionization. The apparent permeability (Pₐₚₚ) were calculated as follows:

Pₐₚₚ=(dCᵣ/dt)×Vᵣ/(A×C_{N})

With dCᵣ/dt being the slope of the cumulative concentration in the recover compartment versus time in ny Ms⁻¹;

Vᵣ is the volume of the receiver compartment in cm³

A is the area of the insert (1.13 cm² for 12-well)

C_{N} is the nominal concentration of the dosing solution in my M. The results are presented in Table 2 below.

TABLE 2

| | | Recovery | Papp | |
|---|---|---|---|---|
| Comp. | Structure | (%) | (10-6 cm/s) | Log P |
| E2 | | 15 | 9.42 | 4.13 |
| EE | | 59 | 42.5 | 4.52 |
| EC537 | 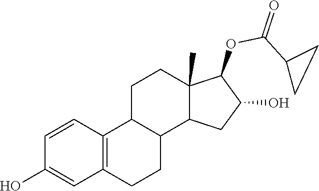 [SMILES: C[C@]12CCC3c4ccc(O)cc4CCC3C1C[C@@H](O)[C@@H]2OC(=O)C1CC1] | 26 | 24.0 | 3.55 |
| | MW: 356.46 | | | |
| EC5104 | 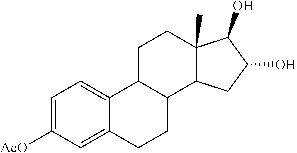 [SMILES: CC(=O)Oc1ccc2c(c1)CCC1C2CC[C@@]2(C)C1C[C@@H](O)[C@@H]2O] | 64 | 23.7 | 3.02 |
| | Formula Weight: 330.42 | | | |
| EC5105 | 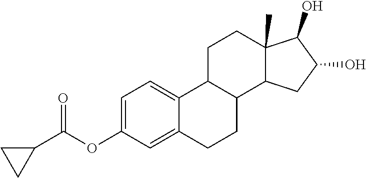 [SMILES: C[C@]12CCC3c4ccc(OC(=O)C5CC5)cc4CCC3C1C[C@@H](O)[C@@H]2O] | 3 | 1.76 | 3.38 |
| | Formula Weight: 356.46 | | | |
| EC5106 | 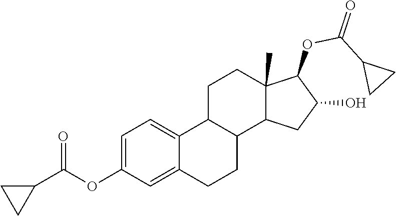 [SMILES: C[C@]12CCC3c4ccc(OC(=O)C5CC5)cc4CCC3C1C[C@@H](O)[C@@H]2OC(=O)C1CC1] | 25 | 11.4 | 3.99 |
| | Formula Weight: 424.53 | | | |

### Example 11: Solubility Determination

Solubilities were determined by shaking (24 hours) an excess of steroid in 20 ml of water/medium at 37° C. Following equilibrium, a portion was passed through a 0,22m-my filter and the steroid concentration in the filtrate was determined by high-performance liquid chromatography. Solubilities in EVA were estimated by hot stage microscopy. The results are presented in Table 3 below.

TABLE 3

| | Solubility in | Solubility in water + | Solubility in | |
|---|---|---|---|---|
| | water at 37° C. | 2% SLS at 37° C. | EVA28/2.5 at 37° C. | |
| API | (μg/mL) | (μg/mL) | (via HSM) | |
| EC357 | 4.2 | 3296 | 4-5 | wt % |
| EC5104 | 57.8 | 5361 | 0.5 | wt % |
| EC5105 | 4.9 | 3097 | 0.5-2 | wt % |

| | | | |
|---|---|---|---|
| E3 | 58.4 | 465 | 0.5-1% |

### Discussion

There are just three vaginal ring products releasing estrogenic compounds on the market: NUVARING, releasing 0.015 mg ethinyl estradiol per day; FEMRING, releasing 0.0075 mg estradiol per day; and ESTRING, releasing 0.05 to 0.1 mg estradiol acetate per day. It is noteworthy to mention, that for accomplishing a daily release of 0.1 mg estradiol, the ESTRING device uses a more lipophilic prodrug of estradiol, namely the estradiol 3-acetate.

Estriol is a natural estrogen and its use is especially desirable since it offers significant advantages over synthetic estrogens (e.g., ethinyl estradiol and estradiol) when it comes to safety in indications like contraception and menopause management. Some of the advantages of estriol are: (a) lack of hepatic estrogenicity; (b) no stimulatory effect on breast tissue; (c) less induction of bleeding episodes than estradiol in postmenopausal women.

Estriol, however, offers a significant challenge when it comes to securing therapeutic plasma levels over the whole cycle based on the short half-life, the low solubility in thermoplastic polymers and the high doses that need to be delivered daily based on the lower intrinsic activity of estriol compared to estradiol and ethinyl estradiol. For example, estriol shows a 10 to 30% reduction in activity compared to estradiol depending on the model applied. Estradiol needs to be applied in doses ranging from 0.05 to 0.1 mg/day when given as a vaginal ring (Estring®). Therefore, it can be assumed that for estriol, having much weaker activity than estradiol, daily doses of 0.15 to 1.00 mg could be anticipated.

Estradiol release rates in this range couldn't be accomplished from a thermoplastic matrix because of the low solubility of estradiol in polymers. Necessary plasma levels were reached by using the 3-acetate ester of estradiol instead of estradiol as taught in European Patent No. 0 799 025 (EP '025). EP '025 described the investigation of a range of mono- and diesters of estradiol and came to the following conclusion. The daily release rate of estradiol could be increased around 3-fold by using the 17-acetate derivative of estradiol, whereas the daily release rate of the 3-acetate turned out to be above 45 times higher than the release rate seen with estradiol.

The challenge to deliver therapeutic plasma levels of estriol are significantly higher compared to estradiol, based on the differences in the physicochemical properties, and the different potencies. Estriol has a log P of 2.81 compared to a log P of estradiol of 3.94, indicating a much higher lipophilicity of estradiol.

It can be assumed that daily estriol release rates between 0.15 and 1.00 mg are needed to reach sufficient plasma levels. In order to achieve such high release rates, the teaching of EP '025 was applied to estriol.

When determining the solubility in water none of the investigated prodrugs showed a higher solubility than the parent molecule estriol, whereas the estradiol-3-acetate showed a twofold higher solubility in water. Similar differences between the estradiol and estriol esters were observed when it comes to the solubility in polymers. Estradiol-3-acetate exhibits an around 10 fold higher solubility in silicone than estradiol, whereas the solubility of the estriol-3-acetate are comparable to the estriol solubility in EVA. Results of the Caco-2 investigations also showed unexpected results. The more lipophilic diesters showed a significantly lower permeability than the respective monoester.

In addition opposite to the teaching of EP '025, the estriol 3-ester of cyclpropyl carbonic acid showed a very low permeability compared to the 17 analog.

In summary, it can be concluded from the in vitro data that the results of EP '025 suggest that hydrocarbon ester derivatives could not be applied to estriol derivatives at all.

It was therefore quiet surprising and totally unexpected that some of the investigated prodrugs showed significantly higher plasma levels of estriol after being released from vaginal rings in sheep. EC537 lead to around 10 fold higher plasma levels of estriol compared to estriol its self. In summary, it was demonstrated that unexpectedly, the 17-ester of estriol with cyclopropyl carboxylic acid showed a significantly higher, i.e., an around 10-fold, daily release rate than the estriol-3-acetate as proposed by EP '025.

### Intravaginal Drug Delivery

As used herein, an “intravaginal device” refers to an object that provides for administration or application of an active agent to the vaginal and/or urogenital tract of a subject, including, e.g., the vagina, cervix, or uterus of a female.

In an embodiment, an intravaginal drug delivery device includes one or two or more compartments joined to each other. Each of the compartments includes an estrogen prodrug and/or a progestin. Each compartment may be an uncoated polymeric matrix that includes the active agent or a coated polymeric matrix that includes the active agent. A combination of coated and uncoated compartments may be combined to form a ring-shaped drug delivery device.

A variety of materials may be used as the matrix for the compartments. Generally, the compartments used in the intravaginal device are suitable for extended placement in the vaginal tract or the uterus. In an embodiment, a thermoplastic material is used to form the intravaginal drug delivery device. The thermoplastic material is nontoxic and non-absorbable in the subject. In some embodiments, the materials may be suitably shaped and have a flexibility allowing for intravaginal administration.

In a preferred embodiment, compartments of an intravaginal drug delivery device are formed from an ethylene vinyl acetate copolymer (EVA). A variety of grades may be used including grades having a low melt flow index, a high melt flow index, a low vinyl acetate content or a high vinyl acetate content. As used herein, EVA having a “low melt flow index” has a melt flow index of less than about 100 g/10 min as measured using ASTM test 1238. EVA having a “high melt flow index” has a melt index of greater than about 100 g/10 min as measured using ASTM test 1238. EVA having a “low vinyl acetate content” has a vinyl acetate content of less than about 20% by weight. EVA having a “high vinyl acetate content” has a vinyl acetate content of greater than about 20% by weight. The compartments of the intravaginal drug delivery device may be formed from EVA having a low melt flow index, a high melt flow index, a low vinyl acetate content or a high vinyl acetate content. In some embodiments, the thermoplastic matrix may include: mixtures of a low melt flow index and high melt flow index EVA or mixtures of low vinyl acetate content and high vinyl acetate content EVA.

In an embodiment, the thermoplastic polymeric matrix comprises an ethylene vinyl acetate copolymer. In an embodiment, the thermoplastic polymeric matrix comprises a thermoplastic polyurethane. In an embodiment, the compartment is a coated compartment that includes a thermoplastic polymeric matrix comprising an ethylene vinyl acetate (EVA) copolymer with a VA (vinyl acetate) content between 18% and 40% and wherein the coating comprises an EVA copolymer with a VA content between 6% and 18%. In another embodiment, the thermoplastic polymeric matrix includes an ethylene vinyl acetate (EVA) copolymer with a VA (vinyl acetate) content between 18% and 40% in the core and a low-density polyethylene (LDPE).

In an embodiment, a combination of one or more suitable materials may be used to form the compartments. The material(s) may be selected to allow prolonged release of the active ingredients from the compartment. In addition, the concentration of the active agents, in combination with the matrix material may be selected to provide the desired release from the compartment. In some compartments, a coating may be applied to the matrix to yield reservoir systems to further control the release rate of the active ingredients. The coating may be formed from the same material, or a different material than the thermoplastic matrix used to form the compartment.

In one embodiment, the compartment may be composed of ethylene vinyl acetate copolymer in combination with the hydrophobic polymer hydroxy propyl cellulose.

In an embodiment, the active agents, for example the progestin and/or estrogen prodrug, are dispersed in the thermoplastic matrix to form a compartment. As used herein the term “dispersed”, with respect to a thermoplastic matrix, means that a compound is substantially evenly distributed through the polymer, either as a solid dispersion in the polymer or dissolved within the polymer matrix. The term “particle dispersion,” as used herein refers to a dispersion of the compound particles homogenously distributed in the polymer. The term “molecular dispersion,” as used herein refers to the dissolution of the compound in the polymer. For purposes of this disclosure, a dispersion may be characterized as a particle dispersion if particles of the compound are visible in the polymer at a magnification of about 100-fold under regular and polarized light.

A molecular dispersion is characterized as a dispersion in which substantially no particles of the compound are visible in the polymer at a magnification of 100-fold under regular and polarized light.

In an embodiment, the intravaginal drug delivery device is used to produce a contraceptive state in a female mammal. The contraceptive state may be produced by administering an intravaginal drug delivery device that includes a progestin. In other embodiments, contraceptive state may be produced by administering an intravaginal drug delivery device that includes a progestin and an estrogen component.

The intravaginal delivery device can be in any shape suitable for insertion and retention in the vaginal tract without causing undue discomfort to the user. For example, the intravaginal device may be flexible. As used herein, “flexible” refers to the ability of an intravaginal drug delivery device to bend or withstand stress and strain without being damaged or broken. For example, an intravaginal delivery device may be deformed or flexed, such as, for example, using finger pressure, and upon removal of the pressure, return to its original shape. The flexible properties of the intravaginal drug delivery device are useful for enhancing user comfort, and also for ease of administration to the vaginal tract and/or removal of the device from the vaginal tract.

In an embodiment, the intravaginal drug delivery device may be annular in shape. As used herein, “annular” refers to a shape of, relating to, or forming a ring. Annular shapes suitable for use include a ring, an oval, an ellipse, a toroid, and the like. The intravaginal drug delivery device may have a non-annular geometry.

In one embodiment, the intravaginal drug delivery device has a geometry in the form of a strand of geometrically shaped compartments linked together. For example, a plurality of hexagon shaped compartments may be linked to form a strand. Other geometrically shaped units including, but not limited to, squares, triangles, rectangles, pentagons, heptagons, octagons, etc. may be formed into strands. In some embodiment, mixtures of different geometrically shaped units may be joined to together in a strand. The strand of geometrically shaped units may be joined together to form ring-like structure.

In another embodiment, an intravaginal drug delivery device is in the shape of a half oval. A half oval device may be easier to manufacture than a full ring. In an embodiment, the half oval shape may allow a user to form a ring like structure before and/or after insertion. In another embodiment, an intravaginal drug delivery device may be in the shape of a hollow cylinder. Use of a hollow cylinder may allow easier insertion of the intravaginal delivery device. The hollow cylinder geometry may allow insertion of the intravaginal drug delivery device into the vaginal tract in a compressed form, which, upon deployment, expands inside the tract to improve the retention of the device. In another embodiment, an intravaginal drug delivery device may have a monolithic film geometry. Such a film may be formed or include, mucoadhesive substances to improve adhesion to the vaginal tract.

The intravaginal drug delivery device may be manufactured by any known techniques. In some embodiments, therapeutically active agent(s) may be mixed within the thermoplastic matrix material and processed to the desired shape by: injection molding, rotation/injection molding, casting, extrusion, or other appropriate methods. In one embodiment, the intravaginal drug delivery device is produced by a hot-melt extrusion process.

In one embodiment, a method of making an intravaginal drug delivery device includes:
a. forming a mixture of a thermoplastic polymer and the active agent;
b. heating the thermoplastic polymer/active agent mixture such that at least a portion of the thermoplastic polymer is softened or melted to form a heated mixture of thermoplastic polymer and active ingredient;
c. permitting the heated mixture to cool and solidify as a solid mass,
d. and optionally, shaping the mass into a predetermined geometry.

For purposes of the present disclosure a mixture is “softened” or “melted” by applying thermal or mechanical energy sufficient to render the mixture partially or substantially completely molten. For instance, in a mixture that includes a matrix material, “melting” the mixture may include substantially melting the matrix material without substantially melting one or more other materials present in the mixture (e.g., the therapeutic agent and one or more excipients). For polymers, a “softened” or “melted” polymer is a polymer that is heated to a temperature at or above the glass transition temperature of the polymer. Generally, a mixture is sufficiently melted or softened, when it can be extruded as a continuous rod, or when it can be subjected to injection molding.

The mixture of the thermoplastic polymer and the active agent can be produced using any suitable means. Well-known mixing means known to those skilled in the art include dry mixing, dry granulation, wet granulation, melt granulation, high shear mixing, and low shear mixing.

Granulation generally is the process wherein particles of powder are made to adhere to one another to form granules, typically in the size range of 0.2 to 4.0 mm. Granulation is desirable in pharmaceutical formulations because it produces relatively homogeneous mixing of different sized particles.

Dry granulation involves aggregating powders with high compressional loads. Wet granulation involves forming granules using a granulating fluid including either water, a solvent such as alcohol or water/solvent blend, where this solvent agent is subsequently removed by drying. Melt granulation is a process in which powders are transformed into solid aggregates or agglomerates while being heated. It is similar to wet granulation except that a binder acts as a wetting agent only after it has melted. The granulation is further achieved following using milling and/or sieving to obtain the desired particle sizes or ranges. All of these and other methods of mixing pharmaceutical formulations are well-known in the art.

Subsequent or simultaneous with mixing, the mixture of thermoplastic polymer and the active agent is softened or melted to produce a mass sufficiently fluid to permit shaping of the mixture and/or to produce melding of the components of the mixture. The softened or melted mixture is then permitted to solidify as a substantially solid mass. The mixture can optionally be shaped or cut into suitable sizes during the softening or melting step or during the solidifying step. In some embodiments, the mixture becomes a homogeneous mixture either prior to or during the softening or melting step. Methods of melting and molding the mixture include, but are not limited to, hot-melt extrusion, injection molding and compression molding.

Hot-melt extrusion typically involves the use of an extruder device. Such devices are well-known in the art. Such systems include mechanisms for heating the mixture to an appropriate temperature and forcing the melted feed material under pressure through a die to produce a rod, sheet or other desired shape of constant cross-section. Subsequent to or simultaneous with being forced through the die the extrudate can be cut into smaller sizes appropriate for use as an oral dosage form. Any suitable cutting device known to those skilled in the art can be used, and the mixture can be cut into appropriate sizes either while still at least somewhat soft or after the extrudate has solidified. The extrudate may be cut, ground or otherwise shaped to a shape and size appropriate to the desired oral dosage form prior to solidification, or may be cut, ground or otherwise shaped after solidification. In some embodiments, an oral dosage form may be made as a non-compressed hot-melt extrudate. In other embodiments, an oral dosage form is not in the form of a compressed tablet.

Injection molding typically involves the use of an injection-molding device. Such devices are well-known in the art. Injection molding systems force a melted mixture into a mold of an appropriate size and shape. The mixture solidifies as least partially within the mold and then is released.

Compression molding typically involves the use of a compression-molding device. Such devices are well-known in the art. Compression molding is a method in which the mixture is optionally preheated and then placed into a heated mold cavity. The mold is closed and pressure is applied. Heat and pressure are typically applied until the molding material is cured. The molded oral dosage form is then released from the mold.

The final step in the process of making intravaginal drug delivery device is permitting the mixture to solidify as a solid mass. The mixture may optionally be shaped either prior to solidification or after solidification. Solidification will generally occur either as a result of cooling of the melted mixture by different methods (air, water bath) or as a result of curing of the mixture however any suitable method for producing a solid dosage form may be used.

When combining compartments to form an intravaginal drug delivery device, individual compartments may be joined directly together or may be coupled to each other through a spacer formed form a thermoplastic matrix material. The spacer may be formed from the same thermoplastic material used to form the compartments, or may be formed from a different material. The spacer, in some embodiments, does not include any active agents.

Through the use of different compartments in the drug delivery device, the device releases the active ingredients such that each of the released active ingredients has a different non-zero order release kinetic profile, and the amounts of active ingredients released are not constant but rather changing over time. Such release profiles are especially useful in the field of contraception and menopause management.

In one embodiment, a combination of compartments is selected to create release profiles that mimic hormone profiles of regular female cycle, with estrogen being more dominate in the first half, and progestin being more dominate in the second half of the cycle. In some embodiments, compartments may be selected to enable delivery of high concentrations of a progestin, which is responsible for ovulation inhibition, from the first day of treatment to avoid further growth of the leading follicle that has grown in the hormone free interval between two cycles. The timing of the delivery of the appropriate amounts of progestin with the appropriate estrogen ensures a good bleeding profile.

In another preferred embodiment the estrogen prodrug is an estriol prodrug and the progestin is trimegestone.

In one embodiment, an intravaginal drug delivery system includes one or more compartments, each of the compartments including progestin and/or estrogen prodrug embedded in a thermoplastic polyethylene vinyl acetate copolymer. The progestin and/or estrogen prodrug may be either fully dissolved or in a crystalline stage. Each compartment may be an uncoated matrix of thermoplastic polyethylene vinyl acetate copolymer with the active agent(s) dispersed throughout the core. In some embodiments, a compartment may be a coated matrix having a thermoplastic polyethylene vinyl acetate copolymer covering the core.

The individual compartments, may be welded together to form a ring-shaped drug delivery system by using a thermoplastic polymer spacer to link the compartments together. The spacers may be formed from a polyethylene vinyl acetate copolymer capable of inhibiting the exchange of estrogens and progestins from one compartment to the other.

One significant advantage of the intravaginal drug delivery devices described herein is that targeted release profiles can be generated by either: varying the size of the compartments (e.g., the length); varying the loading of active agents (e.g., the progestin or estrogen prodrug); adding a coating material to the compartment; or using a combination of any of these modifications.

Release kinetics identify the drug release process via mathematical models to drug release process (the amount of drug release per unit time). Release kinetics can also be defined by the ratio of active agent released on Day 1 to active agent release on the last day of administration (Day 21 or Day 28). For supersaturated systems where cₒ (initial concentration at t₀) is above the cₛ (saturation concentration), release can also be fitted using the Korsmeyer-Peppas equation, where the drug fraction dissolved at a time, equivalent to active agent release, as a function of time is plotted. The diffusional exponent “n” of the power law and thereby, the drug release mechanism from different polymeric controlled delivery systems for different geometries (thin films, spheres or cylinders) can be determined via the slope of the linear regression fit. The release kinetics follows zero order release (Case-II transport), when the drug release is constant over time (ratio of releases Day 1 to Day 28 is 1) and independent of concentration. For cylinders, a diffusional exponent n of 0.89 or above indicates Case-II Transport and hence, zero order release.

The target release kinetics of a non-zero order release is provided for Day 1/Day 21 (or Day 28) ratios between 1.5 and 4.0. In the Korsmeyer-Peppas equation, non-zero order or anomalous transport (a combination of Case-II transport and Fickian diffusion) is achieved when the diffusional exponent n is between 0.89 and 0.45. A diffusional exponent of 0.45 indicates Fickian diffusion.

In preferred embodiments, the compartments include an active agent as a substantially uniform dispersion within a thermoplastic matrix. In alternative embodiments the distribution of the active agent within the thermoplastic matrix can be substantially non-uniform. One method of producing a non-uniform distribution of the active agent is through the use of one or more coatings of water-insoluble or water-soluble polymers. Another method is by providing two or more mixtures of polymer or polymer and the active agent to different zones of a compression or injection mold. These methods are provided by way of example and are not exclusive.

In practice, for a human female, an annular intravaginal drug delivery device has an outer ring diameter from 35 mm to 70 mm, from 35 mm to 60 mm, from 45 mm to 65 mm, or from 50 mm to 60 mm. The cross-sectional diameter may be from 1 mm to 10 mm, from 2 mm to 6 mm, from 3.0 mm to 5.5 mm, from 3.5 mm to 4.5 mm, or from 4.0 mm to 5.0 mm.

The release rate can be measured in vitro using compendial methods, e.g., the USP Apparatus Paddle 2 method, or a rotational incubation shaker. The active agent(s) can be assayed by methods known in the art, e.g., by HPLC or UPLC.

In some embodiments of the present invention, active agent(s) is/are released from the intravaginal device for up to about 1 month or about 28 days after administration to a female, for up to about 25 days after administration to a female, for up to about 21 days after administration to a female, for up to about 15 days after administration to a female, for up to about 10 days after administration to a female, for up to about 7 days after administration to a female, or for up to about 4 days after administration to a female. In embodiments intended for contraceptive use, the device delivers an effective amount of the progestin and the estrogen prodrug for at least 21 days.

Each individual compartment may release an active agent at a steady rate. As used herein, a “steady rate” is a release rate that does not vary by an amount greater than 70% of the amount of active agent released per 24 hours in situ, by an amount greater than 60% of the amount of active agent released per 24 hours in situ, by an amount greater than 50% of the amount of active agent released per 24 hours in situ, by an amount greater than 40% of the amount of active agent released per 24 hours in situ, by an amount greater than 30% of the amount of active agent released per 24 hours in situ, by an amount greater than 20% of the amount of active agent released per 24 hours in situ, by an amount greater than 10% of the amount of active agent released per 24 hours in situ, or by an amount greater than 5% of the amount of active agent released per 24 hours in situ.

In some embodiments, the active agent is trimegestone with a compartment steady release rate of active agent in situ of about 80 μg to about 200 μg per 24 hours, about 90 μg to about 150 μg per 24 hours, about 90 μg to about 125 μg per 24 hours, or about 95 μg to about 120 μg per 24 hours.

In some embodiments, the active agent is estriol prodrug with a compartment steady release rate of active agent in situ of about 50 μg to about 800 μg per 24 hours, about 100 μg to about 500 μg per 24 hours, about 150 μg to about 300 μg per 24 hours.

The release kinetics and drug release profile can be impacted by selecting the type of system. Reservoir systems are designed to yield zero order release kinetics (Case-II transport), whereas matrix systems provide either Fickian diffusion (drug release proportional to surface and drug loading) or anomalous transport (combination of Fickian diffusion and Case-II transport). For reservoir systems, release rates can be modulated by the skin thickness and type of polymer used. EVA copolymers with high vinyl acetate (VA) content show reduced crystallinity and hence, increased permeability, whereas EVA polymers with low VA content yield increased crystallinity and hence, reduced permeability.

In some embodiments, the active agent is released according to a non-zero order release, where the ratio of active agent release Day 1 to Day 21/28 is in the range of 1.5-4.0, more specifically, the ratio is in the range of 1.5-3.0, even more specifically, in the range of 1.5-2.0.

In some embodiments, the active agent is released according to anomalous transport (a combination of Case-II transport and Fickian diffusion). This refers to a diffusional exponent (in the Korsmeyer-Peppas Equation) for cylinders of 0.89-0.45.

In some embodiments, the drug delivery rate may be characterized by measuring the amount of progestin and/or estrogen prodrug released on the last day of treatment. In one embodiment, the amount of progestin and/or estrogen prodrug released by the device on the last day of treatment is at least 50% higher than on any day after the first day of use.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An intravaginal drug delivery device comprising:
one or more compartments, each of the one or more compartments comprising an estrogen prodrug and/or a progestin dispersed in a thermoplastic polymeric matrix.

2. The device of claim 1, wherein one or more of the compartments are uncoated compartments.

3. The device of claim 1, wherein one or more of the compartments are coated compartments comprising an estrogen prodrug and/or progestin dispersed in a coated thermoplastic polymeric matrix.

4. The device of claim 1, wherein the device comprises two or more compartments having different sizes.

5. The device of claim 1, wherein the estrogen prodrug is a mono or di ester of estriol.

6. The device of claim 1, wherein the estrogen prodrug is a prodrug of estriol having structure: 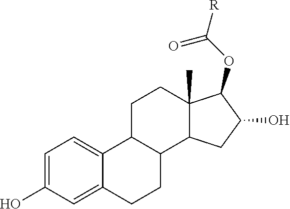
where R is a saturated hydrocarbon.

7. The device of claim 6, wherein R is methyl.

8. The device of claim 6, wherein R is cyclopropyl.

9. The device of claim 1, wherein the estrogen prodrug is a prodrug of estriol having the structure: 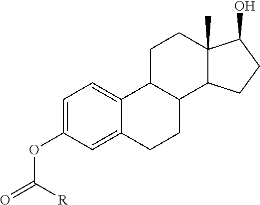
where R is a saturated hydrocarbon.

10. The device of claim 9, wherein R is methyl.

11. The device of claim 9, wherein R is cyclopropyl.

12. The device of claim 1, wherein the estrogen prodrug is a prodrug of estriol having the structure: 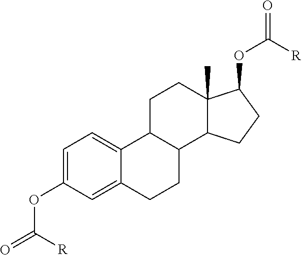
where R is a saturated hydrocarbon.

13. The device of claim 12, wherein R is cyclopropyl.

14. The device of claim 1, wherein the device comprises at least one compartment comprising a progestin, and wherein the progestin is released, during vaginal use, in an amount sufficient to inhibit ovulation in fertile women.

15. The device of claim 14, wherein the progestin is trimegestone.

16. The device of claim 1, wherein the device comprises at least one compartment comprising an estrogen prodrug and at least one compartment comprising a progestin, and wherein the estrogen prodrug and progestin are released, during vaginal use, in amounts sufficient to effect cycle control in fertile women.

17. The device of claim 1, wherein the intravaginal drug delivery device provides the estrogen prodrug and/or the progestin according to a non-zero order release profile.

18. The device of claim 1, wherein the thermoplastic polymeric matrix comprises an ethylene vinyl acetate copolymer.

19. The device of claim 1, wherein the thermoplastic polymeric matrix comprises a thermoplastic polyurethane.

20. The device of claim 1, wherein the compartment is a coated compartment comprising a thermoplastic polymeric matrix comprising an ethylene vinyl acetate (EVA) copolymer with a VA (vinyl acetate) content between 18% and 40% and wherein the coating comprises an EVA copolymer with a VA content between 6% and 18%.

21. The device of claim 1, wherein the thermoplastic polymeric matrix comprises an ethylene vinyl acetate (EVA) copolymer with a VA (vinyl acetate) content between 18% and 40% in the core and a low density polyethylene (LDPE).

22-25. (canceled)

26. The device of claim 1, wherein the amount of progestin and/or estrogen prodrug released by the device on the last day of treatment is at least 50% higher than on any day after the first day of use.
